(19) United States
(12) Patent Application Publication
(10) Pub. No.: US 2004/0106208 A1
(43) Pub. Date: Jun. 3, 2004

(54) Analysis of sulfate using conductometric end-point detection with suppression of cationic co-precipitation

(76) Inventors: Peter M. Robertson, Winkel (CH); Binoi Shah, Menlo Park, CA (US)

Correspondence Address: ATMI, INC., 7 COMMERCE DRIVE, DANBURY, CT, 06810, US

(21) Appl. No.: 10/307,659

(22) Filed: Dec. 2, 2002

(51) Int. Cl. G01N 33/00

(52) U.S. Cl. 436/119

(57) ABSTRACT

The present invention provides for a method of monitoring the concentration of a sulfate salt present in a solution. In accordance with this aspect of the present invention, the concentration of sulfate salt present in the solution is monitored by titrating a known concentration of a barium salt solution with a test sample of the solution containing an unknown amount of sulfate salt under conductivity titration conditions.

### BACKGROUND OF THE INVENTION

1. Field of the Invention

The present invention relates to a titration method, and more particularly, to a conductometric, precipitation titration to determine the concentration of sulfate in a fluid sample while suppressing cationic co-precipitation.

2. Description of the Related Art

Solder bumping, is an advanced microelectronic chip packaging and connection technology used basically to connect chips, chip packages, or such other units by means of solder balls placed between two surfaces of the units (e.g. IBM's Controlled Collapse Chip Connection (C4) Technology). These tiny balls, of electrically conductive solder, bridge the gaps between respective pairs of metal pads on the units being connected.

A major application of solder bumping is in joining semiconductor microchips (integrated circuits) to chip packages. Electrical engineers are constantly placing more and more circuits onto each chip, to improve performance and reduce cost. As the number of circuits on a chip grows, so does the number of connections needed. Solder bumping technology allows for a very high density of electrical connections, and thus, is commercially important.

One method of forming solder bumps is electrochemical fabrication. This method includes a continuous seed layer to provide an electrical path for through-mask electrode deposition of the bumps. The seed layer is deposited and then a layer of photoresist is applied and patterned to create vias. For high-end applications, the solder comprises a lead-tin alloy to form PbSn bumps. Seed layers used in the fabrication of PbSn bumps consists of Cu as a solderable layer, phased CrCu as a glue layer, and a TiW alloy as an adhesion layer. After the seed layer is deposited, the PbSn solder is then electroplated. After electroplating, the photoresist is stripped and the seed layer between the individual solder bump pads is removed by etching.

Etching of the seed layer is a critical processing step and the seed layer has to be completely removed in order to eliminate electrical contact between solder bumps while the remaining seed layer under the bumps act as a solder pad. The seed layer etching consists of both electroetching to remove the Cu layer and chemical etching to remove the TiW layer, both of which must occur without damage to the solder balls.

A typical chemical etching bath to selectively remove TiW layer contains hydrogen peroxide acting as an etchant and a sulfate salt acting as a passivating agent that forms a protective layer over the PbSn bumps. Monitoring of the etching bath is important to insure that the initial concentration of the sulfate salt is maintained and replenished, if required. Further, by measuring the sulfate content accurately, a good estimate of evaporation or drag-out losses can be obtained.

U.S. Pat. No. 6,238,589 to Cooper et al. describes a method for monitoring the concentration of the sulfate salt component of a metal etching bath by titrating the metal etching solution with a barium salt solution under turbidimetric titration conditions. Specifically, the concentration of sulfate is determined by titrating the etchant solution with a titrant comprising a barium salt solution under turbidimetric titration conditions that measure and compare the opaqueness of liquids.

However, the method according to Cooper, et al. introduces an additional complication by co-precipitating cations of the sulfate component, e.g., potassium, which introduces an error of approximately 6% lower than the theoretical result.

U.S. Pat. No. 4,814,281 describes a method for detecting the concentration of trace sulfate in the steam cycle water of fossil or nuclear-fueled power generating plants by conductometric monitoring. This testing method involves titration with barium chloride and passes of the test sample through multiple cation exchange columns. The method further includes adjusting the pH of the water sample with nitric acid to maintain selectivity of barium sulfate precipitation. However, it should be noted that the water-testing sample does not include hydrogen peroxide or the problems that can be introduced by mixing nitric acid and hydrogen peroxide. It has been reported by multiple researchers that at higher concentrations of nitric acid, a reaction between the hydrogen peroxide and nitric acid can occur which may be violent in nature and produce heat and/or large volumes of NOx gas.

Accordingly, there is a need in the art for a method for sulfate concentration analysis that overcomes the shortcomings of the prior art, such as reducing inherent errors in concentration values due to cationic co-precipitation during precipitation titrations and/or the possibility of violent reactions that can produce heat and large volumes of NOx gas.

### SUMMARY OF THE INVENTION

The present invention relates to a method and system for monitoring the concentration of sulfate ions in a process solution (e.g. a metal etching solution), while reducing co-precipitation of sulfate counter-ions to reduce errors in determining the concentration of sulfate in solution.

In one aspect, the present invention relates to a method for determining a sulfate salt concentration in a test sample solution by precipitation titration of sulfate anions without co-precipitation of the sulfate counter-ions; the method comprising:

a) providing a test sample solution comprising at least sulfate anions and sulfate counter-ions;

b) providing a reactive agent solution in a titration vessel, wherein the reactive agent solution comprises a known amount of a precipitatable cation to combine with the sulfate anion and form an insoluble sulfate precipitate,

c) titrating the reactive agent solution with the test sample solution, wherein the sulfate anions of the test sample solution are precipitated in the titration vessel without co-precipitation of the sulfate counter-ions; and

d) measuring a suitable parameter of the reactive agent solution during the addition of the test sample solution until an equivalence point is indicated by that parameter and possible further titration with the test sample solution to effect continuous change in that measured parameter at a different rate than displayed prior to the equivalence point to allow for accurate interpolation of the position of the equivalence point.

In a further aspect, the present invention relates to a method for determining a sulfate salt concentration in a test sample solution by precipitation titration of sulfate anions without co-precipitation of the sulfate counter-ions; the method comprising:

a) providing a test sample solution comprising at least sulfate anions and sulfate counter-ions;

b) providing a reactive agent solution in a titration vessel, wherein the reactive agent solution comprises a known amount of a precipitatable cation to combine with the sulfate anion and form an insoluble sulfate precipitate,

c) titrating the reactive agent solution with the test sample solution, wherein the sulfate anions of the test sample solution are precipitated in the titration vessel without co-precipitation of the sulfate counter-ions; and

d) measuring the conductivity of the reactive agent solution during the addition of the test sample solution until an equivalence point is reached and further titration with the test sample solution causes a change in conductivity.

In another aspect, the present invention relates to a method for determining a potassium sulfate concentration in a metal etching solution by precipitation titration of sulfate anions without co-precipitation of the potassium counter-ions, the method comprising:

a) providing a test sample solution having an unknown concentration of the potassium sulfate;

b) providing a known volume and concentration of a barium salt solution in a titration vessel, wherein the known concentration of the barium salt solution stoichiometically equals or exceeds the initial concentration of potassium sulfate in the test sample solution;

c) measuring the initial conductivity of the barium salt solution;

d) titrating the barium salt solution with the test sample solution, wherein the sulfate in the test sample solution is precipitated as an insoluble barium salt while co-precipitation of the potassium counter-ions is reduced; and

e) measuring the conductivity of the barium salt solution during the addition of the test sample solution while a change in conductivity is occurring at a first given rate until an equivalence point is reached and an increase of the sulfate anions and potassium counter-ions in the titration vessel causes a change in conductivity at a second and different current rate.

In a still further aspect, the present invention relates to a monitoring system for determining the concentration of a potassium sulfate salt in a test sample of a metal etching solution by precipitation titration without cationic co-precipitation; the system comprising:

a) a titration vessel for containing a known volume and concentration of a barium salt solution;

b) a sample input port for introducing a test sample of the metal etching solution to the monitoring system;

c) a burette communicatively connected to the titration vessel;

d) a collection loop communicatively connected to the sample input port, the titration vessel and burette for displacing the test sample from the sample input port to the burette and then to titration vessel; and

e) means for measuring conductivity communicatively connected to the titration vessel to determine the conductivity of the barium salt solution during the titration of the barium salt solution with the test sample of metal etching solution.

Other aspects and features of the invention will be more fully apparent from the ensuing disclosure and appended claims.

### BRIEF DESCRIPTION OF THE DRAWINGS

FIGS. 1A and 1B graphically illustrate ratios of potassium ions to barium ions during a titration method of the prior art and the present invention, respectively.

FIG. 2 is a graph showing an end point determination for the titration of a barium chloride solution with the metal etching solution containing a potassium sulfate salt.

FIG. 3 illustrates a schematic arrangement of components in the sulfate analyzing system of the present invention.

FIG. 4 is a graph showing an end point determination for the titration of a barium chloride solution with a metal etching solution containing a potassium sulfate salt

### DETAILED DESCRIPTION OF THE INVENTION AND PREFERRED EMBODIMENTS THEREOF

The present invention provides for a method of monitoring and replenishing the concentration of a sulfate salt present in a metal etching solution. In accordance with this aspect of the present invention, the concentration of the sulfate salt present in the metal etchant solution is monitored by titrating a known concentration of a barium salt solution with a test sample of the metal etching solution containing an unknown amount of the sulfate salt under conductivity titration conditions. The concentration of the sulfate salt is determined and compared to the initial sulfate salt concentration in the metal etching solution and additional sulfate salt may be added to the metal etching solution to bring the concentration back to its initial concentration.

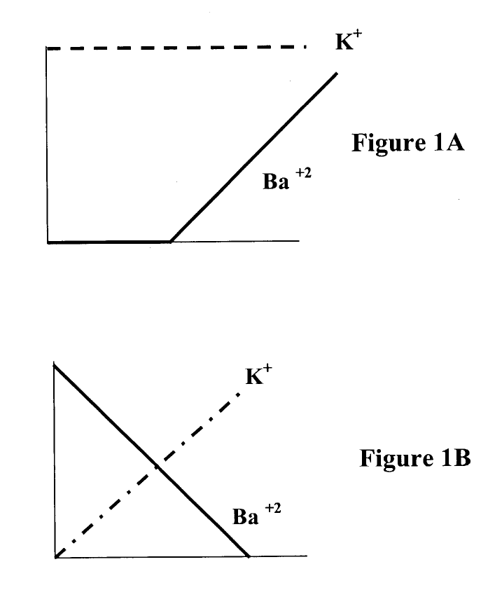

Previous methods for determining the quantity of a sulfate salt in a metal etching solution include titration of a metal etching solution with a barium salt titrant. However, the previous methods typically resulted in an approximately 6% error in the sulfate concentration relative to the higher theoretical value. It is believed, by the present inventors, that this error is due to co-precipitation of the potassium counter-ion that occurs in a solution comprising a higher concentration of potassium ions relative to barium ions during the titration. During the titration of the metal etching solution, using the previous methods, the barium salt titrant is immediately precipitated as barium sulfate, so essentially during the entire titration up to the end-point there is a negligible concentration of barium in solution. Thus, through most of the titration there is a high ratio of potassium ions to barium ions, as shown in FIG. 1A.

The present invention obviates the above-mentioned problem by providing a method wherein the concentration of barium ions compound is higher than the concentration of potassium ions during at least half of the titration process, thus reducing the likelihood of co-precipitation of the sulfate counter-ion, such as the potassium.

The present invention corrects for this approximately 6% error by reorganization of the titration procedure. It has been found that determining the concentration of the sulfate under the conditions of a low potassium ion concentration and a high barium ion concentration the co-precipitation error can be reduced and even eliminated. Thus, instead of titrating the test sample containing the sulfate anion with the barium salt titrant, the titration is performed in reverse, that being, titrating a given amount of a barium salt solution with the metal etching solution used as the titrant. Using the present method, there is an excess of barium ions relative to the potassium ions at the start of the titration, such as shown in FIG. 1B and the insoluble barium sulfate is precipitated without co-precipitation of potassium ions.

The present invention uses traditional chemistry for precipitating sulfate anions as barium sulfate. Further, the concentration of the sulfate ions present in the metal etchant solution is determined by titrating a known excess amount of a soluble barium salt with a sample of the metal etching solution under conductometric titration conditions. By conductometric titration conditions it is meant a titration technique which is capable of measuring and comparing the conductivity of a barium salt solution before commencing the titration, during the titration and after reaching an equivalence point using a sample of the metal etching solution containing a sulfate salt as the titrant. A visible end point is can be determined by plotting a conductivity curve generated during the titration process.

The term “equivalence point” as used herein means that all substance are present in the amounts required for a complete reaction.

The term “end-point” as used herein means the point in the titration when some effect occurs such as a color change or noticeable change in conductivity or rate of change of conductivity indicating that no more titrant should be added. The end point generally occurs slightly before or after the equivalence point.

The term “sulfate salt” as used herein means sulfate-containing compounds which include a metal from Group I of the Periodic Table of Elements, i.e., sodium, potassium, and the like. An ammonium salt, or protonated amine salt, are also contemplated herein. Of these sulfate salts, it is highly preferred that potassium sulfate be present in the metal etchant solution.

The barium salt solution is preferably BaCl₂. Other possible choices include Ba(NO₃)₂ and Ba(ClO₄)₂.

The metal etching solution used as the titrant in the present invention is generally a combination of a sulfate salt and at least hydrogen peroxide. The sulfate anion in the titrant reacts with the barium salt solution in the titration vessel as in the following reaction.

K₂SO₄+BaCl₂→BaSO₄↓2 KCl

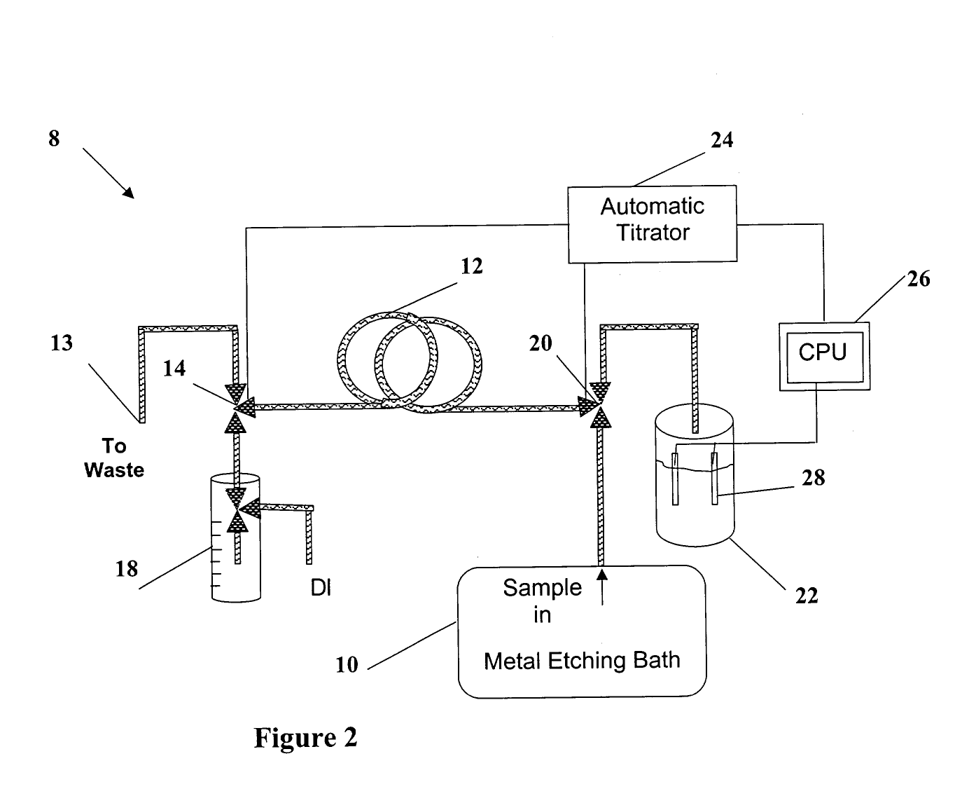

A suitable arrangement 8 for collecting the test sample and dispensing accurately to the titration vessel is shown in FIG. 2. A test sample of the metal etching solution comprising an unknown concentration of a sulfate salt is flushed from the etching bath 10 through the sample collection loop 12 to waste 13 to remove from the loop any remaining solution from a previous testing. Valves 14 and 16 on the loop system are activated and a precision burette 18 displaces a test sample from the loop. The test sample is sent to the titration vessel 22 via valve 20.

The titration vessel 22 contains a known volume and concentration of a barium salt solution that is titrated with the test sample of the metal etching solution for determination of the concentration of the sulfate anion therein. Preferably, the concentration of the barium salt placed in the titration vessel is approximately stoichiometically equal to the concentration of the sulfate salt initially included in the metal etchant solution. Using the approximate stoichiometric equal concentration assures there will be sufficient barium cations to precipitate essentially all sulfate anions in the test sample solution and to ensure that an accurate end-point is reached.

The titration vessel 22 further includes a conductometric detection system, which may comprise a pair of inert electrodes, such as a pair of platinum electrodes 28 placed in series with a microampmeter. A small constant voltage can be placed across the electrodes, however, because the barium salt solution is conductive and produces at least a minimal current, this is not required for an accurate determination of the sulfate concentration.

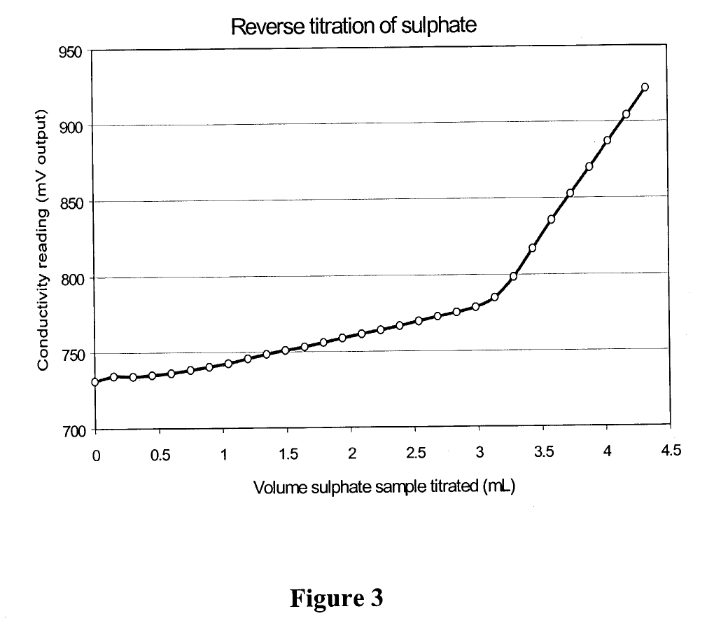

The titration can be controlled by an automatic titrator 24, which controls the precision burette and the amount of titrant that is flowed to the titration vessel. As the titrant, containing the sulfate anion, is introduced into the titration vessel, the current between the electrodes slowly increases, at a particular rate, as the barium cations are precipitated as a sulfate salt and replaced with an increasing amount of potassium cations in the titration vessel. At the time when all the barium sulfate is precipitated and the titrant of the metal etching solution is momentarily continued, the excess of the potassium sulfate in addition to the potassium chloride in the titration vessel causes a highly conductive solution with an observably increasing current, at a second rate, as shown on the microampmeter. The end point of the reaction is very abrupt as shown in FIG. 3 wherein microamps are plotted against the titrant volume delivered in milliliters. This abrupt change in the conductance measurement is sensed, but reagent is titrated further. The data before and after the abrupt change in the conductance measurement is used to interpolate the exact position of the end-point. A central processing unit 26 may analyze the data to trigger the automatic titrator to close off the titrant flow from the burette 18 to the titration vessel.

The electrical conductivity change curve is obtained from a change of electrical conductivity in the barium salt solution with the amount of metal etching solution titrant introduced into the titration vessel and the electrical conductivity is detected by means of an electrically conductivity sensor communicatively connected to the electrodes. A signal obtained by the use of the electrodes can be in the form of a voltage-change, so that it can be converted into an electric current for use in the measurement of electrical conductivity therein.

In the above-described manner, the electrical conductivity change can be continuously detected from the start of the titration until the completion of the chemical reaction (equivalence point is reached) in the titration vessel to obtain the electrical conductivity curve for the entire titration process. The end point on the conductivity graph, which generally occurs slightly before and/or after the equivalence point, can be easily observed as the sharp intersection of two essentially straight lines. The end point of the reaction can be very abrupt as shown in FIG. 3, wherein microamps are plotted against the titrant volume delivered in milliters. This very strong and detectable end point is illustrated for a typical titration made in accordance with the present invention.

The conductivity may be recorded on any suitable recording means, such as a convention strip chart recorder, and subsequently analyzed by comparison with a table of conductivity differences versus known sulfate ion concentrations. FIG. 3 illustrates conductivity results utilizing a known volume and concentration of a barium salt solution in the titration vessel with a constant flow of the metal etching solution titrant until the equivalence point is reached and an end-point indicates same. The horizontal axis represents the volume of titrant added versus the current generated until the end point is detected.

Determination of the concentration of the sulfate in the testing sample can be measured by several different methods. For instance, analyses can be performed by comparing the end-point conductivity with conductivity values found by intentional additions of varying concentrations of sulfate added to a known volume and concentration of a barium chloride solution and extrapolation therefrom.

A standard value may be obtained by comparing the measured conductivity of a solution containing barium chloride after the addition of a known amount of a testing sample with a standard value. The standard value may be conveniently obtained by measuring the conductivity of a similar solution of barium chloride containing no testing sample. Alternatively, the standard may be calculated according to theoretical principles from the characteristics of the conductivity cell and the concentration, charge and mobility factor of the ions in solution.

The difference in the conductivity, i.e., conductivity differential, is a direct function of the amount of sulfate in the testing sample and provides a method of increased sensitivity. The electrical conductivity of a liquid is dependent not only on the concentration of the ions in solution but also on the mobility of the ion and conductivity increases with ion mobility. Thus, the mobility of ions in solution, and consequently their conductivity varies with changes in temperature. Generally, an increase in temperature will increase the mobility of the ions, and thus, the conductivity of a given liquid will increase as it temperature is raised. Accordingly, it may be desirable to control the temperature of a liquid sample when making conductivity measurements to determine the concentration of the sulfate component in the liquid.

The features, aspects and advantages of the present invention are further shown with reference to the following non-limiting examples relating to the invention.

### EXAMPLES

Comparison of Methods for Sulfate Concentration Determinations Determination of Sulfate Concentration Using Conductometric End-Point Detection and Reverse Titration

1. Reagent (barium chloride) and sample (potassium sulfate) were transferred to titration vessel via precision burettes. The sample used contained potassium sulfate and other chemicals including hydrogen peroxide. The predetermined concentration of potassium sulfate was 185.2 g/l.

Experimental

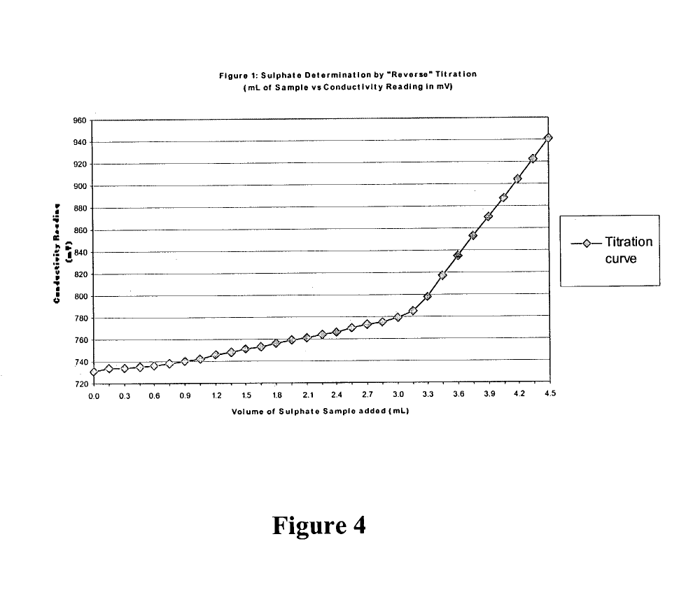

A standard solution of barium chloride was prepared in an empty titration vessel by diluting 6.6 ml of 0.5 mol/l BaCl2 to a standard volume with DI water. While being continuously stirred the standard solution was tritrated with a known volume of sample. A conductivity probe was used to measure the conductivity of the solution and the results are shown in FIG. 4. The end point of the titration was determined by the crossing of two best-fit lines for the two distinct regions of the curve.

The results of eight determinations using the method described above are shown Table

TABLE 1
Summary of Sulfate Analysis Using Reverse Titration

| | Volume of Sulphate | Calculated |
|---|---|---|
| Run | sample used (ml) | K2SO4 [g/l] |
| 1 | 3.10 | 185.4 |
| 2 | 3.12 | 184.4 |
| 3 | 3.11 | 184.8 |
| 4 | 3.11 | 184.7 |
| 5 | 3.10 | 185.6 |
| 6 | 3.11 | 184.8 |
| 7 | 3.11 | 184.6 |
| 8 | 3.11 | 184.8 |
| | Average | 184.9 |

2. Prior Art Method for Determination of Sulfate Concentration

Reagent (barium chloride) and sample (potassium sulfate) were transferred to titration vessel via precision burettes. The sample used contained potassium sulfate and other chemicals including hydrogen peroxide. The predetermined concentration of potassium sulfate was 190.6 g/l.

Experimental

A test solution was prepared by transferring 3 ml of sample to an empty titration vessel, and diluting to a standard volume with DI water. While being continuously stirred the standard solution was tritrated with a known volume of 0.5 mol/l barium chloride. A conductivity probe was used to measure the conductivity of the solution.

Table 2 shows the results of eight determinations using the prior art method described above, having an error of 6%.

TABLE 2
Summary of Sulfate Analysis Using Prior Art Method

| | Volume of 0.5 mol/l | Calculated |
|---|---|---|
| Run | Barium Chloride used (ml) | K2SO4 [g/l] |
| 1 | 6.176 | 179.3 |
| 2 | 6.172 | 179.2 |
| 3 | 6.181 | 179.4 |
| 4 | 6.181 | 179.5 |
| 5 | 6.151 | 178.6 |
| 6 | 6.154 | 178.7 |
| 7 | 6.167 | 179.0 |
| 8 | 6.167 | 179.0 |
| | Average | 179.1 |

What is claimed is:

1. A method for determining a sulfate salt concentration in a test sample solution by precipitation titration of sulfate anions without co-precipitation of the sulfate counter-ions; the method comprising:
a) providing a test sample solution comprising at least sulfate anions and sulfate counter-ions;
b) providing a reactive agent solution in a titration vessel, wherein the reactive agent solution comprises a known amount of a precipitatable cation to combine with the sulfate anion and form an insoluble sulfate precipitate,
c) titrating the reactive agent solution with the test sample solution, wherein the sulfate anions of the test sample solution are precipitated in the titration vessel without co-precipitation of the sulfate counter-ions; and
d) measuring a suitable parameter of the reactive agent solution during the addition of the test sample solution until an equivalence point is indicated by that parameter and possible further titration with the test sample solution to effect continuous change in that measured parameter at a different rate than displayed prior to the equivalence point to allow for accurate interpolation of the position of the equivalence point.

2. The method according to claim 1, wherein the parameter used to detect the equivalence point is conductivity.

3. A method for determining a sulfate salt concentration in a test sample solution by precipitation titration of sulfate anions without co-precipitation of the sulfate counter-ions; the method comprising:
a) providing a test sample solution comprising at least sulfate anions and sulfate counter-ions;
b) providing a reactive agent solution in a titration vessel, wherein the reactive agent solution comprises a known amount of a precipitatable cation to combine with the sulfate anion and form an insoluble sulfate precipitate,
c) titrating the reactive agent solution with the test sample solution, wherein the sulfate anions of the test sample solution are precipitated in the titration vessel without co-precipitation of the sulfate counter-ions; and
d. measuring the conductivity of the reactive agent solution during the addition of the test sample solution until an equivalence point is reached and further titration with the test sample solution causes a change in conductivity.

4. The method according to claim 3, wherein the conductivity of the reactive agent solution during the addition of the test sample solution changes at a first rate, and the conductivity after the equivalence point changes at a second and different current rate.

5. The method according to claim 3, wherein the reactive agent is a barium salt.

6. The method according to claim 3, wherein the sulfate salt is potassium sulfate, sodium sulfate or ammonium sulfate.

7. The method according to claim 3, wherein the abrupt switch in rate of change in conductivity is the endpoint of the precipitation titration.

8. The method according to claim 3, wherein the conductivity curve is compared to conductivity curves prepared from known concentrations of the sulfate containing solution.

9. The method according to claim 8, wherein the conductivity curve is a curve of conductivity versus volume addition of the sulfate containing solution.

10. The method according to claim 5, wherein half of the titration is conducted with an excess of barium salt in the reactive agent solution.

11. The method according to claim 3, wherein the test sample solution is a metal etching solution comprising potassium sulfate, hydrogen peroxide and a soluble EDTA salt.

12. The method according to claim 5, wherein the test sample solution is added to the barium salt solution at a rate of about 0.5 to about 2 μL/sec.

13. The method according to claim 5, wherein the barium salt is barium chloride, barium nitrate or barium chlorate.

14. A method for determining a potassium sulfate concentration in a metal etching solution by precipitation titration of sulfate anions without co-precipitation of the potassium counter-ions, the method comprising:
a) providing a test sample solution having an unknown concentration of the potassium sulfate;
b) providing a known volume and concentration of a barium salt solution in a titration vessel, wherein the known concentration of the barium salt solution stoichiometically equals or exceeds the initial concentration of potassium sulfate in the test sample solution;
c) measuring the initial conductivity of the barium salt solution;
d) titrating the barium salt solution with the test sample solution, wherein the sulfate in the test sample solution is precipitated as an insoluble barium salt while co-precipitation of the potassium counter-ions is reduced; and
e) measuring the conductivity of the barium salt solution during the addition of the test sample solution until an equivalence point is reached and an increase of the sulfate anions and potassium counter-ions in the titration vessel causes a change in conductivity.

15. The method according to claim 14, further comprising determining the sulfate anion concentration in the test sample solution from a conductivity curve generated during the titration.

16. The method according to claim 15, wherein the barium salt is barium chloride, barium nitrate or barium chlorate.

17. The method according to claim 14, wherein the abrupt change in conductivity is the end-point of the precipitation titration.

18. The method according to claim 15, wherein the conductivity curve is a curve of current generated versus volume addition of the test sample solution comprising anions and cations.

19. A monitoring system for determining the concentration of a sulfate salt in a test sample of a metal etching solution by precipitation titration without cationic co-precipitation; the system comprising:
a) a titration vessel for containing a known volume and concentration of a barium salt solution;
b) a sample input port for introducing a test sample of the metal etching solution to the monitoring system;
c) a burette communicatively connected to the titration vessel;
d) a collection loop communicatively connected to the sample input port, the titration vessel and burette for displacing the test sample from the sample input port to the burette and then to titration vessel; and
e) means for measuring conductivity communicatively connected to the titration vessel to determine the conductivity of the barium salt solution during the titration of the barium salt solution with the test sample of metal etching solution.
